(19) United States
(12) Patent Application Publication
(10) Pub. No.: US 2023/0363987 A1
(43) Pub. Date: Nov. 16, 2023

(54) Oral Care Compositions

(71) Applicant: Colgate-Palmolive Company, New York, NY (US)

(72) Inventors: Rong DONG, Highland Park, NJ (US); Suman CHOPRA, Monroe, NJ (US); Gregory SZEWCZYK, Flemington, NJ (US)

(73) Assignee: Colgate-Palmolive Company, New York, NY (US)

(21) Appl. No.: 18/212,478

(22) Filed: Jun. 21, 2023

Related U.S. Application Data
(63) Continuation of application No. 17/887,237, filed on Aug. 12, 2022, now abandoned.
(60) Provisional application No. 63/232,985, filed on Aug. 13, 2021.

(51) Int. Cl. A61K 8/22; A61K 8/21; A61K 8/34; A61K 8/90; A61Q 11/00

(52) U.S. Cl. CPC A61K 8/22; A61K 8/21; A61K 8/345; A61K 8/90; A61Q 11/00; A61K 2800/28; A61K 2800/30; A61K 2800/48

(57) ABSTRACT

Described herein are oral care compositions comprising: from about 35 wt. % to about 85 wt. % of an anhydrous solvent system (e.g., 35 wt. % to about 75 wt. %); from about 0.02 wt. % to about 30 wt. % of a peroxide solution comprising: from about 0.01 wt. % to about 15 wt. % of hydrogen peroxide, and from about 0.01 wt. % to about 15 wt. % of water; from about 1 wt. % to about 20 wt. % of a thickening system; wherein the oral care composition has a total water content of less than about 30 wt. %; and wherein all weight percentages are based on the total weight of the oral care composition. Methods of making and using these compositions are also described herein.

### CROSS-REFERENCE TO RELATED APPLICATIONS

This application claims the benefit of priority to U.S. Provisional Patent Application Ser. No. 63/232,985 filed Aug. 13, 2021, the contents of which are incorporated herein by reference in its entirety.

### BACKGROUND

Many individuals desire a “bright” smile and white teeth, and consider dull and stained teeth cosmetically unattractive. Thus, there is a desire for whiter teeth and one means to achieve whiter teeth is the use of tooth whitening products.

Teeth can become discolored by foods, drinks and tobacco use. Dental stains can be classified as either extrinsic, which occur on the outer surface of teeth, or intrinsic, which occur below the surface of enamel. Most abrasive containing toothpaste remove extrinsic stains. Peroxides can bleach both extrinsic and intrinsic stains and so provides fast and superior whitening efficacy. The peroxide can bleach the teeth, remove stains, and kill cariogenic bacteria.

However, peroxide compounds are highly reactive, and consequently difficult to formulate. In particular, hydrogen peroxide (H₂O₂) has been conventionally avoided due to the numerous manufacturing and stability difficulties resulting from the reactivity of hydrogen peroxide. For instance, hydrogen peroxide can spontaneously decompose to form molecular oxygen (O₂) and water. The off-gas accumulation can be problematic in storage, causing dentifrice containers to bloat, burst, or leak. Additionally, the degradation of hydrogen peroxide in dentifrice compositions may cause and the remaining formulation to not have enough remaining peroxide to clean and whiten teeth effectively.

To alleviate this, some dentifrices initially comprise very high levels of peroxide, which decomposes over time, so that the exact amount of peroxide delivered on application is variable and largely depends on how long and under what conditions the dentifrice has been stored. In alternative methods, the pH of the composition is decreased to an acidic range in order to increase H₂O₂ stability.

Current oral care products do not adequately address off-gassing resulting from hydrogen peroxide decomposition. Accordingly, there is a need for oral care compositions having stable hydrogen peroxide formulations. Accordingly, embodiments of the present invention are designed to provide these, and other, benefits.

### BRIEF SUMMARY

The present disclosure relates to oral care composition and methods of producing and using the same. In accordance with some aspects of the invention, provided is an oral care composition including from about 35 to about 75 wt. % of a polyol; from about 0.02 to about 30 wt. % of a peroxide solution comprising: from about 0.01 to about 15 wt. % of hydrogen peroxide, and from about 0.01 to about 15 wt. % of water; from about 1 to about 20 wt. % of a polymer; and from about 0.5 to about 10 wt. % of an anionic surfactant, wherein the oral care composition has a total amount of water of up to 25 wt. % and a pH of 4.5 or more, wherein all weight percentages are based on the total weight of the oral care composition.

In some embodiments, the oral care composition further includes a fluoride ion source. The amount of fluoride ion source may be from about 0.1 to about 4 wt. %. Suitable fluoride ion sources include amine fluoride, an alkali metal fluoride salt, a monofluorophosphate salt (e.g., sodium monofluorophosphate), and an ion thereof.

Additionally, or alternatively, the oral care composition may have from 1 to about 30 wt. % of an abrasive. The abrasive may be selected from silica (e.g., wherein the silica is not a high cleaning silica), alumina, insoluble phosphates, calcium carbonate, resinous abrasives, and a combination of two or more thereof.

Non-limiting examples of polyols that may be incorporated into the oral care composition include ethylene glycol, propylene glycol, butylene glycol, hexylene glycol, pentylene glycol, 1,3-propanediol, diethylene glycol, dipropylene glycol, caprylyl glycol, glycerin, or a combination of two or more thereof. In at least one embodiment, the polyol comprises propylene glycol.

In one aspect, the oral care composition may comprise a hydrogen peroxide solution having a weight ratio of the hydrogen peroxide to the water from about 1:5 to about 5:1. For instance, the weight ratio of the hydrogen peroxide to the water is about 1:2 to about 2:1. In at least one embodiment, the weight ratio of the hydrogen peroxide to the water is about 1:1. For example, the hydrogen peroxide solution can include products marketed under the trade name: PERSYNT 500 Super D, PERSYNT 500 Alkali and PERSYNT 500 Cosmetic by Evonik; PEROXAL 35 CG, PEROXAL 50 CG from Arkema; or INTEROX from Solvay. As used herein, “peroxide solution” and “hydrogen peroxide solution” are used interchangeably.

In one aspect, the oral care composition can have a weight ratio of total water to the polymer comprising polyvinyl pyrrolidone from about 1:1 to about 1:30. In some embodiments, the oral care composition has a weight ratio of the total water to polymer comprising polyvinyl pyrrolidone from about 1:1 to about 1:20.

In one aspect, the oral care composition of the disclosure can comprises one or more anionic surfactant(s) may be selected from sodium lauryl sulfate, sodium lauryl sarcosinate, sodium methyl cocoyl taurate, sodium monoglyceride sulfate, sodium cetaryl sulfate, potassium cocoyl glycinate, sodium lauryl phosphate, sodium lauryl lactylate, sodium lauryl sulfoacetate, sodium lauryl glutamate, sodium lauryl isethionate, sodium laureth carboxylate, sodium dodecyl benzenesulfonate, and a combination of two or more thereof.

In certain aspects, the oral care composition may be formulated to have a pH from 4.5 to about 9 In some instances, the oral care composition comprises about 0.01 to about 12 wt. % of water, optionally about 0.01 to about 10 wt. % of water, optionally about 0.01 to about 8 wt. % of water, optionally about 0.01 to about 6 wt. % of water, or optionally about 0.01 to about 4 wt. % of water. In one embodiment, the oral care composition is substantially free of a non-incidental amount of water or, optionally, is free of added water.

In some aspects the oral care composition may comprise about 5 wt. % or less, optionally about 4 wt. % or less, optionally about 3 wt. % or less, optionally about 2 wt. % or less, or optionally about 1 wt. % or less, of a fatty compound. In some instances, the oral care composition is substantially free of fatty compounds or, optionally, is free of fatty compounds.

According to another aspect of the invention, provided is a kit. The kit typically includes a container and an oral care composition disposed in the container.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other apparatuses and methods. Before explaining the disclosed embodiments of the present invention in detail, it is to be understood that the invention is not limited in its application to the details of any particular embodiment shown. The terminology used herein is for the purpose of description and not of limitation.

Unless otherwise specified, all percentages and amounts expressed herein and elsewhere in the specification should be understood to refer to percentages by weight relative to the total composition. The amounts given are based on the active weight of the material unless specified otherwise.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2.0 wt. %” refers to a number between and including 1.800 wt. % and 2.200 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the oral care composition. The symbol “°” refers to a degree, such as a temperature degree or a degree of an angle. The symbols “h”, “min”, “mL”, “nm”, “μm” means hour, minute, milliliter, nanometer, and micrometer, respectively. The abbreviation “UV-VIS” as referring to a spectrometer or spectroscopy, means Ultraviolet-Visible. The abbreviation “rpm” means revolutions per minute.

As used herein, “high cleaning silica” can refer to silica having a pellicle cleaning ratio (PCR) of greater than 85 when tested at 20% loading as is accepted in the art as high cleaning silica. Typically, high cleaning silica also has a mean particle size d₅₀ of from 5 to 15 μm and an oil absorption of from 40 to 120 cm³/100 g silica. Examples of high cleaning silica include silica marketed under the brand name ZEODENT® 105 from Evonik.

When referring to chemical structures, and names, the symbols “C”, “H”, and “O” mean carbon, hydrogen, and oxygen, respectively. The symbols “—”, “═” and “≡” mean single bond, double bond, and triple bond respectively.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the oral care compositions of the instant disclosure can be free or essentially free of all components and elements positively recited throughout the instant disclosure. In some instances, the oral care compositions of the present disclosure may be substantially free of non-incidental amounts of the ingredient(s) or compound(s) described herein. A non-incidental amount of an ingredient or compound is the amount of that ingredient or compound that is added into the oral care composition by itself. For example, an oral care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the oral care composition includes both components (or the composition includes more than two components that overlap), an overlapping compound does not represent more than one component. For example, certain compounds may be characterized as both an emulsifier and a surfactant. If a particular oral care composition includes both an emulsifier and a surfactant, a compound that may be characterized as both an emulsifier and a surfactant will serve only as either an emulsifier or a surfactant—not both.

For readability purposes, the chemical functional groups are in their adjective form; for each of the adjective, the word “group” is assumed. For example, the adjective “alkyl” without a nouns thereafter, should be read as “an alkyl group”.

Aspects of the present invention relate to oral care compositions and methods of making and using the same. The inventors discovered that oral care compositions having certain ingredients in particular weight ratios surprisingly have an enhanced stability for hydrogen peroxide in the presence of water and at a pH of 4.5 or greater. For example, the oral care compositions disclosed herein may exhibit about 5% or less of the hydrogen peroxide degradation when stored at a temperature of 40° C. for 1 month. Preferably, about 4% or less, about 3% or less, about 2% or less, about 1% or less, or about 0.5% or less of the hydrogen peroxide present in the oral care composition degrades when stored at a temperature of 40° C. for 1 month.

Surprisingly, the oral care compositions may exhibit the enhanced peroxide stability in the presence of water at a pH of 4.5 or greater. In some instances, the oral care compositions stably comprise hydrogen peroxide in the presence of water at a pH of 4.5 to about 10, 4.5 to about 9, 4.5 to about 8, 4.5 to about 7, 4.5 to about 6; about 5 to about 10, about 5 to about 9, about 5 to about 8, about 5 to about 7, about 5 to about 6; about 6 to about 10, about 6 to about 9, about 6 to about 8, about 6 to about 7; about 7 to about 10, about 7 to about 9, or about 7 to about 8, including any ranges and subranges therebetween.

The oral care compositions of the disclosure may, advantageously, be produced using a peroxide solution, such as an aqueous hydrogen peroxide solution. In some cases, the hydrogen peroxide solution may have a weight ratio of hydrogen peroxide to water of about 10:1 to about 1:50, about 10:1 to about 1:10, or about 1:2 to about 2:1, e.g., 1:1. Additionally or alternatively, in a further aspect, the oral care composition may be formulated to have a weight ratio of hydrogen peroxide solution to polyvinyl pyrrolidone of about 1:1 to about 1:30.

In certain aspects, the oral care compositions of the disclosure, may comprise about 5 wt. % or less of fatty compounds, based on the total weight of the oral care composition. In some embodiments, the oral care composition comprises about 4 wt. % or less, optionally about 3 wt. % or less, optionally about 2 wt. % or less, or optionally about 1 wt. % or less, of a fatty compound, based on the total weight of the oral care composition. In a further embodiment, the oral care composition is substantially free or free of fatty compounds. The fatty compounds may be fatty alcohols, fatty esters, fatty ethers, fatty oils (e.g., hydrocarbon oils), derivatives thereof, and/or a combination of two or more thereof.

Additionally or alternatively, in still a further aspect, the oral care composition may be formulated to have a viscosity from about 50,000 to 250,000 centipoise (cP), about 70,000 to 225,000 centipoise (cP), about 100,000 to 200,000 centipoise (cP), including all values in between these ranges, at room temperature using Brookfield viscometer and a spindle no. 3.

The oral care compositions of the disclosure may be in the form of, e.g., a dentifrice (e.g., a toothpaste), or mouthwash. Suitable components, such as those listed below, may be included or excluded from the formulations for the oral care compositions depending on the specific combination of other ingredients and the form of the oral care compositions. In a further aspect the oral care compositions of the disclosure be in a form selected from the following: dentifrice (e.g., toothpaste), a mouthwash or a mouth rinse, a topical oral gel, a denture cleanser, spray, toothpaste powder, tablet, mousse, foam, and chewing gum.

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may also be used in nonhuman mammals such as companion animals (e.g., dogs and cats), as well as by humans. In some embodiments, the oral care compositions as disclosed herein are used by humans. Examples of such compositions include, but are not limited to, toothpaste or a dentifrice, a mouthwash or a mouth rinse, a topical oral gel, a denture cleanser, sprays, toothpaste powders, tablets, mousse, foam, chewing gums and the like.

As used herein, the term “dentifrice” means paste, gel, or liquid formulations unless otherwise specified. The dentifrice composition can be in any desired form, e.g., deep striped, surface striped, multi-layered, having the gel surrounding the paste, or any combination thereof. Alternatively, in one aspect, the oral composition may be dual phase dispensed from a separated compartment dispenser.

In some aspects, the present invention provides an oral care composition comprising: an anhydrous solvent system typically in a range from about 35 to about 75 wt. %, based on the total weight of the oral care composition. For example, the amount of anhydrous solvent system present in the oral care composition may be from about 35 to about 75 wt. %, about 40 to about 75 wt. %, about 45 to about 75 wt. %, about 50 to about 75 wt. %; about 35 to about 65 wt. %, about 40 to about 65 wt. %, about 45 to about 65 wt. %, about 50 to about 65 wt. %; about 35 to about 60 wt. %, about 40 to about 60 wt. %, about 45 to about 60 wt. %, or about 50 to about 60 wt. %, including ranges and subranges therebetween, based on the total weight of the oral care composition.

The anhydrous solvent system may comprise one or more compounds having a hydroxyl group. For example, the anhydrous system may include a mono alcohol or a polyol. Suitable polyols include glycols, glycerin, and block copolymers, such as those comprising or consisting of ethylene oxide and/or propylene oxide. In some embodiments, the anhydrous system includes one or more of a block copolymer of ethylene oxide and propylene oxide; ethylene glycol, propylene glycol, butylene glycol, hexylene glycol, pentylene glycol, 1,3-propanediol, diethylene glycol, polyethylene glycol; a polyethylene glycol/polypropylene glycol copolymer; dipropylene glycol, caprylyl glycol, and glycerin. In further embodiments, the anhydrous solvent system comprises a solvent selected from: propylene glycol; a polyethylene glycol/polypropylene glycol copolymer; a block copolymer of ethylene oxide and propylene oxide; glycerin; sorbitol; and a combination of two or more thereof.

In a further aspect, the one or more polyols may be chosen from polyols having from 2 to 15 carbon atoms and at least two hydroxyl groups. Non-limiting examples of polyols that may be incorporated in the oral care composition include ethylene glycol, propylene glycol, butylene glycol, hexylene glycol, pentylene glycol, 1,3-propanediol, diethylene glycol, dipropylene glycol, caprylyl glycol, glycerin, and a mixture thereof. The polyol may be a glycol, such as those chosen from ethylene glycol, propylene glycol, butylene glycol, hexylene glycol, pentylene glycol, diethylene glycol, dipropylene glycol, caprylyl glycol, and a combination of two or more thereof.

Additionally, or alternatively, the polyol may be a glycol ether. Examples of glycol ethers include monomethyl, monoethyl, and monobutyl ethers of ethylene glycol, propylene glycol or ethers thereof such as, e.g., monomethyl ether of propylene glycol, butylene glycol, hexylene glycol, dipropylene glycol as well as alkyl ethers of diethylene glycol, e.g., monoethyl ether or monobutyl ether of diethylene glycol. Other polyols of mention include alkanediols, such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol. In at least one embodiment, the polyol is propylene glycol.

In some aspects, the oral care composition of the disclosure may comprise polymers and/or copolymers of polyethylene glycol, of ethylene oxide/propylene oxide, and of silicone. If such copolymers/polymers are used, they may be selected from commercially available materials. In some embodiments, such block copolymer is an ethylene oxide, propylene oxide block co-polymer of formula (ethylene oxide)ₓ-(propylene oxide)_{y} wherein x is an integer of 80-150, e.g., 100-130, e.g., about 116 or about 118, and y is an integer 30-80, e.g., about 60-70, e.g., about 66, having an average molecular weight of greater than 5000 Da, e.g., 8000-13000 Da, e.g., about 9800 Da. An illustrative ethylene oxide, propylene oxide block co-polymer is PLURACARE® L1220 (available from BASF, Wyandotte, Mich., United States of America). In some embodiments, the ethylene oxide, propylene oxide block co-polymer is present in an amount of from 5% to 15%, e.g., from 6% to 9%, from 7% to 8%, or about 7.5%, by weight of the composition.

In a further aspect, the oral care composition of the present disclosure may comprise a block copolymer of ethylene oxide (EO) and propylene oxide (PO). The block copolymers of ethylene oxide and propylene oxide may be nonionic. For example, the block copolymers of ethylene oxide and propylene oxide may be a nonionic surfactant. The block copolymers of ethylene oxide and propylene oxide may be represented by formula (1).

(ethylene oxide)ₓ−(propylene oxide)_{y}−(ethylene oxide)_{z} (1)
where x may be an integer of from about 5 to about 15 (e.g., x=9-13, or about 11), y may be an integer from about 10 to about 20 (e.g., y=13-17, or about 16), and z may be an integer from about 5 to about 15 (e.g., x=9-13, or about 11). In a certain embodiment, the block copolymer of ethylene oxide and propylene oxide may be represented by formula (2).

(ethylene oxide)₁₁−(propylene oxide)₁₆−(ethylene oxide)₁₁ (2)

The block copolymer of ethylene oxide and propylene oxide may have an average molecular weight of from about 1,000 Da to about 3,000 Da. For example, the block copolymer of ethylene oxide and propylene oxide may have an average molecular weight of from about 1,000 Da, about 1,100 Da, about 1,200 Da, about 1,300 Da, about 1,400 Da, about 1,500 Da, about 1,600 Da, about 1,700 Da, about 1,800 Da, or about 1,850 Da to about 1,950 Da, about 2,000 Da, about 2,100 Da, about 2,200 Da, about 2,300 Da, about 2,400 Da, about 2,500 Da, about 2,600 Da, about 2,700 Da, about 2,800 Da, about 2,900 Da, or about 3,000 Da. In another example, the block copolymer of ethylene oxide and propylene oxide may have an average molecular weight of from about 1,000 Da to about 2,800 Da, about 1,100 Da to about 2,700 Da, about 1,200 Da to about 2,600 Da, about 1,300 Da to about 2,500 Da, about 1,400 Da to about 2,400 Da, about 1,500 Da to about 2,300 Da, about 1,600 Da to about 2,200 Da, about 1,700 Da to about 2,100 Da, about 1,800 Da to about 2,000 Da, or about 1,850 Da to about 1,950 Da. In some aspects, the block copolymer of ethylene oxide and propylene oxide may have an average molecular weight of from about 1,850 Da to about 1,950 Da, e.g., about 1,900 Da.

In one aspect, illustrative block copolymers of ethylene oxide (EO) and propylene oxide (PO) may be or include, but are not limited to, PLURONIC® L35, PLURONIC® LI, PLURONIC® L43, PLURONIC® L10, PLURONIC® L44, PLURONIC® 10R5, PLURONIC® 17R4, PLURONIC® L25R4, PLURONIC® P84, PLURONIC® P65, PLURONIC® PI 04, PLURONIC® PI 05, and the like, and combinations thereof, all of which are commercially available from BASF of Mount Olive, NJ. In a certain embodiment, the block copolymer of ethylene oxide and propylene oxide is PLURONIC® L35.

In further aspects, if the oral care composition of the disclosure comprises a block copolymer of ethylene oxide and propylene oxide then it is present in an amount from 40% to 60% by weight of the composition.

In some aspects, the oral care compositions may include hydrogen peroxide obtained from a hydrogen peroxide solution. In some aspects, the oral care composition of the disclosure comprises hydrogen peroxide, wherein the hydrogen peroxide is obtained as an aqueous solution comprising hydrogen peroxide and water. In some aspects, the hydrogen peroxide solution may have a weight ratio of hydrogen peroxide to water from about 10:1 to about 1:50. In some cases, the hydrogen peroxide aqueous solution has a weight ratio of hydrogen peroxide to water from about 10:1 to about 1:40, about 10:1 to about 1:30, about 10:1 to about 1:20, about 10:1 to about 1:10, about 10:1 to about 1:8, about 10:1 to about 1:6, about 10:1 to about 1:4, about 10:1 to about 1:2, about 8:1 to about 1:50, about 6:1 to about 1:50, about 4:1 to about 1:50, about 2:1 to about 1:50, including ranges and subranges therebetween, such as about 10:1 to about 1:10, about 8:1 to about 1:8, about 6:1 to about 1:6, about 4:1 to about 1:4, about 1:3 to about 2:1, about 2:1 to about 1:2, or about 1:1. In further aspects, the hydrogen peroxide aqueous solution has a weight ratio of hydrogen peroxide to water from about 1:10 to about 10:1, about 1:8 to about 10:1, about 1:6 to about 10:1, about 1:4 to about 10:1, about 1:3 to about 10:1, or about 1:2 to about 10:1.

In still a further aspect, the total amount of hydrogen peroxide solution included in the oral care composition may vary depending on the weight ratio of hydrogen peroxide to water. In some embodiments, the amount of hydrogen peroxide solution present in the oral care composition is from about 0.02 to about 30 wt. %, about 0.02 to about 25 wt. %, about 0.02 to about 20 wt. %, about 0.02 to about 18 wt. %, about 0.02 to about 16 wt. %, about 0.02 to about 14 wt. %, about 0.02 to about 12 wt. %, about 0.02 to about 10 wt. %, about 0.02 to about 9 wt. %, about 0.02 to about 8 wt. %, about 0.02 to about 7 wt. %, about 0.02 to about 6 wt. %, about 0.02 to about 5 wt. %, about 0.02 to about 4 wt. %; about 1 to about 15 wt. %, about 1 to about 13 wt. %, about 1 to about 11 wt. %, about 1 to about 10 wt. %, about 1 to about 9 wt. %, about 1 to about 8 wt. %, about 1 to about 7 wt. %, about 1 to about 6 wt. %, about 1 to about 5 wt. %, about 1 to about 4 wt. %, about 3 to about 15 wt. %, about 3 to about 13 wt. %, about 3 to about 11 wt. %, about 3 to about 10 wt. %, about 3 to about 9 wt. %, about 3 to about 8 wt. %, about 3 to about 7 wt. %, about 3 to about 6 wt. %, about 3 to about 5 wt. %, about 3 to about 4 wt. %; about 5 to about 15 wt. %, about 5 to about 13 wt. %, about 5 to about 11 wt. %, about 5 to about 10 wt. %, about 5 to about 9 wt. %, about 5 to about 8 wt. %, about 5 to about 7 wt. %, about 5 to about 6 wt. %; about 6 to about 15 wt. %, about 6 to about 13 wt. %, about 6 to about 11 wt. %, about 6 to about 10 wt. %, about 6 to about 9 wt. %, about 6 to about 8 wt. %, or about 6 to about 7 wt. %, including ranges and subranges therebetween, based on the total weight of the oral care composition.

Additionally, or alternatively, in still a further aspect, the oral care compositions of the disclosure may be formulated to comprise hydrogen peroxide in an amount from about 0.01 to about 15 wt. %, based on the total weight of the oral care composition. For instance, the amount of hydrogen peroxide present in the oral care composition may be from about 0.01 to about 15 wt. %, about 0.01 to about 13 wt. %, about 0.01 to about 11 wt. %, about 0.01 to about 10 wt. %, about 0.01 to about 9 wt. %, about 0.01 to about 8 wt. %, about 0.01 to about 7 wt. %, about 0.01 to about 6 wt. %, about 0.01 to about 5 wt. %, about 0.01 to about 4 wt. %; about 0.1 to about 15 wt. %, about 0.1 to about 13 wt. %, about 0.1 to about 11 wt. %, about 0.1 to about 10 wt. %, about 0.1 to about 9 wt. %, about 0.1 to about 8 wt. %, about 0.1 to about 7 wt. %, about 0.1 to about 6 wt. %, about 0.1 to about 5 wt. %, about 0.1 to about 4 wt. %; about 1 to about 15 wt. %, about 1 to about 13 wt. %, about 1 to about 11 wt. %, about 1 to about 10 wt. %, about 1 to about 9 wt. %, about 1 to about 8 wt. %, about 1 to about 7 wt. %, about 1 to about 6 wt. %, about 1 to about 5 wt. %, about 1 to about 4 wt. %; about 2 to about 15 wt. %, about 2 to about 13 wt. %, about 2 to about 11 wt. %, about 2 to about 10 wt. %, about 2 to about 9 wt. %, about 2 to about 8 wt. %, about 2 to about 7 wt. %, about 2 to about 6 wt. %, about 2 to about 5 wt. %, about 2 to about 4 wt. %; about 3 to about 15 wt. %, about 3 to about 13 wt. %, about 3 to about 11 wt. %, about 3 to about 10 wt. %, about 3 to about 9 wt. %, about 3 to about 8 wt. %, about 3 to about 7 wt. %, about 3 to about 6 wt. %, about 3 to about 5 wt. %, about 3 to about 4 wt. %; about 4 to about 15 wt. %, about 4 to about 13 wt. %, about 4 to about 11 wt. %, about 4 to about 10 wt. %, about 4 to about 9 wt. %, about 4 to about 8 wt. %, about 4 to about 7 wt. %, about 4 to about 6 wt. %, about 4 to about 5 wt. %; about 5 to about 15 wt. %, about 5 to about 13 wt. %, about 5 to about 11 wt. %, about 5 to about 10 wt. %, about 5 to about 9 wt. %, about 5 to about 8 wt. %, about 5 to about 7 wt. %, about 5 to about 6 wt. %; about 6 to about 15 wt. %, about 6 to about 13 wt. %, about 6 to about 11 wt. %, about 6 to about 10 wt. %, about 6 to about 9 wt. %, about 6 to about 8 wt. %, or about 6 to about 7 wt. %, including ranges and subranges therebetween, based on the total weight of the oral care composition.

For example, in a further aspect, the amount of hydrogen peroxide present in the oral care compositions of the disclosure may be from 0.01 to 15 wt. %, 0.01 to 13 wt. %, 0.01 to 11 wt. %, 0.01 to 10 wt. %, 0.01 to 9 wt. %, 0.01 to 8 wt. %, 0.01 to 7 wt. %, 0.01 to 6 wt. %, 0.01 to 5 wt. %, 0.01 to 4 wt. %; 0.1 to 15 wt. %, 0.1 to 13 wt. %, 0.1 to 11 wt. %, 0.1 to 10 wt. %, 0.1 to 9 wt. %, 0.1 to 8 wt. %, 0.1 to 7 wt. %, 0.1 to 6 wt. %, 0.1 to about 5 wt. %, 0.1 to about 4 wt. %; 1 to 15 wt. %, e.g., about 1% or about 2% or about 3% or about 4%, wherein the wt. % is by wt. of the total weight of the oral care composition.

Additionally, or alternatively, in one aspect, the oral care compositions may be formulated to comprise a total amount of water, e.g., in an amount from about 0.01 to about 25 wt. %, based on the total weight of the oral care composition. In one aspect, the total amount of water present in the oral care composition of the disclosure may be about 0.01 to about 20 wt. %, about 0.01 to about 15 wt. %, about 0.01 to about 13 wt. %, about 0.01 to about 11 wt. %, about 0.01 to about 10 wt. %, about 0.01 to about 9 wt. %, about 0.01 to about 8 wt. %, about 0.01 to about 7 wt. %, about 0.01 to about 6 wt. %, about 0.01 to about 5 wt. %, about 0.01 to about 4 wt. %; about 0.1 to about 25 wt. %, about 0.1 to about 20 wt. %, about 0.1 to about 15 wt. %, about 0.1 to about 13 wt. %, about 0.1 to about 11 wt. %, about 0.1 to about 10 wt. %, about 0.1 to about 9 wt. %, about 0.1 to about 8 wt. %, about 0.1 to about 7 wt. %, about 0.1 to about 6 wt. %, about 0.1 to about 5 wt. %, about 0.1 to about 4 wt. %; about 1 to about 25 wt. %, about 1 to about 20 wt. %, about 1 to about 15 wt. %, about 1 to about 13 wt. %, about 1 to about 11 wt. %, about 1 to about 10 wt. %, about 1 to about 9 wt. %, about 1 to about 8 wt. %, about 1 to about 7 wt. %, about 1 to about 6 wt. %, about 1 to about 5 wt. %, about 1 to about 4 wt. %; about 2 to about 25 wt. %, about 2 to about 20 wt. %, about 2 to about 15 wt. %, about 2 to about 13 wt. %, about 2 to about 11 wt. %, about 2 to about 10 wt. %, about 2 to about 9 wt. %, about 2 to about 8 wt. %, about 2 to about 7 wt. %, about 2 to about 6 wt. %, about 2 to about 5 wt. %, about 2 to about 4 wt. %; about 3 to about 25 wt. %, about 3 to about 20 wt. %, about 3 to about 15 wt. %, about 3 to about 13 wt. %, about 3 to about 11 wt. %, about 3 to about 10 wt. %, about 3 to about 9 wt. %, about 3 to about 8 wt. %, about 3 to about 7 wt. %, about 3 to about 6 wt. %, about 3 to about 5 wt. %, about 3 to about 4 wt. %; about 4 to about 25 wt. %, about 4 to about 20 wt. %, about 4 to about 15 wt. %, about 4 to about 13 wt. %, about 4 to about 11 wt. %, about 4 to about 10 wt. %, about 4 to about 9 wt. %, about 4 to about 8 wt. %, about 4 to about 7 wt. %, about 4 to about 6 wt. %, or about 4 to about 5 wt. %, including ranges and subranges therebetween, based on the total weight of the oral care composition. In at least one embodiment, the oral care composition has from about 0.01 wt. % to about 12 wt. % of total water, optionally from about 0.01 wt. % to about 10 wt. % of total water, optionally from about 0.01 wt. % to about 8 wt. % of total water, or optionally from about 0.01 wt. % to about 6 wt. % of total water.

For example, in a further aspect, the oral care compositions of the disclosure may be formulated to comprise a total amount of water, e.g., in an amount from 0.01 to 25 wt. %, based on the total weight of the oral care composition. In one aspect, the total amount of water present in the oral care composition of the disclosure may be 0.01 to 30 wt. %, 0.01 to 20 wt. %, 0.01 to 15 wt. %, 0.01 to 13 wt. %, 0.01 to 11 wt. %, 0.01 to 10 wt. %, 0.01 to 9 wt. %, 0.01 to 8 wt. %, 0.01 to 7 wt. %, 0.01 to 6 wt. %, 0.01 to 5 wt. %, 0.01 to 4 wt. %, by wt. relative to the total weight of the oral care composition.

In a further aspect, the oral care compositions of the disclosure may include a thickening system. The thickening system may comprise one or more polymers. For instance, the thickening system may comprise a polymer selected from polyvinyl pyrrolidone, a polyacrylate, a polymethacrylate, a polyitaconate, an acrylamide, 2-acrylamido-2-methylpropane sulfonic acid (AMPS); and a combination of two or more thereof. The thickening system may include a thickening agent selected from: a polymer; fumed silica; sodium stearate; stearic acid; a fatty amphiphile (e.g. stearyl alcohol or cetearyl alcohol); and a combination of two or more thereof.

In yet another aspect, the oral care compositions of the disclosure may include one or more polymer(s) comprising polyvinyl pyrrolidone. The amount of polymer(s) in the oral care composition may be from about 1 to about 20 wt. %, based on the total weight of the oral care composition. In some instances, the amount of polymer(s) present in the or care composition is from about 1 to about 20 wt. %, about 2 to about 20 wt. %, about 3 to about 20 wt. %, about 4 to about 20 wt. %, about 5 to about 20 wt. %, about 7.5 to about 20 wt. %, about 10 to about 20 wt. %, about 15 to about 20 wt. %; about 1 to about 15 wt. %, about 2 to about 15 wt. %, about 3 to about 15 wt. %, about 4 to about 15 wt. %, about 5 to about 15 wt. %, about 7.5 to about 15 wt. %, about 10 to about 15 wt. %; about 1 to about 10 wt. %, about 2 to about 10 wt. %, about 3 to about 10 wt. %, about 4 to about 10 wt. %, about 5 to about 10 wt. %, about 7.5 to about 10 wt. %; about 1 to about 7.5 wt. %, about 2 to about 7.5 wt. %, about 3 to about 7.5 wt. %, about 4 to about 7.5 wt. %, about 5 to about 7.5 wt. %; about 1 to about 5 wt. %, about 2 to about 5 wt. %, about 3 to about 5 wt. %, about 4 to about 5 wt. %; about 1 to about 4 wt. %, about 2 to about 4 wt. %, about 3 to about 4 wt. %, including all ranges and subranges therebetween, based on the total weight of the oral care composition.

In one aspect, the oral care composition of the disclosure comprises one or more polymer(s), wherein the one or more polymer(s) comprises polyvinyl pyrrolidone. Polyvinyl pyrrolidone generally refers to a polymer containing vinylpyrrolidone (e.g., N-vinylpyrrolidone, N-vinyl-2-pyrrolidione, and N-vinyl-2-pyrrolidinone) as a monomeric unit. The monomeric unit may include a polar imide group, four non-polar methylene groups, and a non-polar methane group. The polyvinyl pyrrolidone may have an average molecular weight in the range 5,000 to 100,000, preferably in the range 5,000 to 50,000. Polyvinyl pyrrolidones that have average molecular weights of 10,000, 30,000 and 40,000 may be commercially available from Sigma Chemjeal Co., GAF Corporation and Sigma Chemical Co. The polyvinyl pyrrolidone may form a hydrogen peroxide-polyvinyl pyrrolidone polymer complexes. Examples of polyvinyl pyrrolidone complexes include those disclosed in U.S. Pat. No. 5,122,370, the contents of which are incorporated herein by reference. In some embodiments, the polymer comprises crosslinked polyvinyl pyrrolidone (crosslinked PVP). In at least one embodiment, the polymer consists of polyvinyl pyrrolidone.

In addition to polyvinyl pyrrolidone, in a further aspect the oral care composition may include a polymer chosen from porous cross-linked polymers, such as polymers of polyvinylpyrrolidone, polyacrylates, a polymethacrylates, a polyitaconates, and an acrylamides. Examples of acrylates that may be included in the oral care composition include, e.g., isobutyl acrylate, tert-butyl acrylate, 2-ethylhexyl acrylate, lauryl acrylate, lauryl/tridecyl acrylate, cetyl acrylate, stearyl acrylate, cyclohexyl acrylate, benzyl acrylate, isobornyl acrylate, 2-methoxyethyl acrylate, 2-ethoxyethyl acrylate, 2-ethoxyethoxyethyl acrylate, 2-phenoxyethyl acrylate, tetrahydrofurfuryl acrylate, 2-hydroxyethyl acrylate, 2-hydroxypropyl acrylate, 4-hydroxybutyl acrylate, dimethylaminoethyl acrylate, 1,4-butanediol acrylate, or a combination of two or more thereof.

The acrylate may be chosen from diacrylates. In some embodiments, the oral care composition includes a diacrylate chosen from 1,4-butanediol, 1,6-hexanediol, tetraethylene glycol, tripropylene glycol, ethoxylated bisphenol-A, and a combination of two or more thereof. Triacrylate monomers include those of: trimethylol propane, ethoxylated, glyceryl propoxy, and pentaerythritol.

Acrylates further include methacrylates, such as methyl methacrylate, ethyl methacrylate, n-butyl methacrylate, isobutyl methacrylate, tert-butyl methacrylate, 2-ethylhexyl methacrylate, lauryl methacrylate, alkyl methacrylate, tridecyl methacrylate, stearyl methacrylate, cyclohexyl methacrylate, benzyl methacrylate, isobornyl methacrylate, 2-hydroxyethyl methacrylate, 2-hydroxypropyl methacrylate, dimethylaminoethyl methacrylate, diethylaminoethyl methacrylate, glycidyl methacrylate, tetrahydrofurfuryl methacrylate, allyl methacrylate, ethylene glycol methacrylate, triethylene glycol methacrylate, tetraethylene glycol methacrylate, 1,3-butyleneglycol methacrylate, 1,6-hexanediol methacrylate, trimethylopropane methacrylate, ethoxyethyl methacrylate and trifluoroethyl methacrylate.

Examples of acrylamides include, but are not limited to, acrylamide, methacrylamide and di(C₁-C₃₀) alkyl-acrylamides and -methacrylamides such as those of methyl, ethyl, propyl, butyl, pentyl, hexyl and the like. N-substituted acrylamides that may be suitable include N-ethylacrylamide, N-tert-butylacrylamide, N-tert-octylacrylamide, N-octylacrylamide, N-decylacrylamide, N-dodecylacrylamide and the corresponding N-substituted methacrylamides. Other N-substituted acrylamides include N-hydroxymethyl acrylamide, N-isopropylacrylamide, N-methylacrylamide, N,N′-methylenebisacrylamide, N-isobutoxymethylacrylamide, N,N-dimethylacrylamide and 2-acrylamido-2-methylpropanesulfonic acid.

In one aspect, the oral care composition of the disclosure may be formulated to have a weight ratio of total water to polymer comprising or consisting of polyvinyl pyrrolidone from about 1:1 to about 1:30. In some cases, the weight ratio of total water to polymer comprising or consisting of polyvinyl pyrrolidone is from about 1:1 to about 1:30, about 1:1 to about 1:28, about 1:1 to about 1:26, about 1:1 to about 1:24, about 1:1 to about 1:22, about 1:1 to about 1:20, about 1:1 to about 1:18, about 1:1 to about 1:16, about 1:1 to about 1:14, about 1:1 to about 1:14, about 1:1 to about 1:12, about 1:1 to about 1:10, about 1:1 to about 1:8, about 1:1 to about 1:6, about 1:1 to about 1:5, about 1:1 to about 1:4, about 1:1 to about 1:3, or about 1:1 to about 1:32, including all ranges and subranges thereof. Additionally or alternatively, the oral care composition may be formulated to have a weight ratio of hydrogen peroxide to polymer comprising or consisting of polyvinyl pyrrolidone from about 1:1 to about 1:30, about 1:1 to about 1:28, about 1:1 to about 1:26, about 1:1 to about 1:24, about 1:1 to about 1:22, about 1:1 to about 1:20, about 1:1 to about 1:18, about 1:1 to about 1:16, about 1:1 to about 1:14, about 1:1 to about 1:14, about 1:1 to about 1:12, about 1:1 to about 1:10, about 1:1 to about 1:8, about 1:1 to about 1:6, about 1:1 to about 1:5, about 1:1 to about 1:4, about 1:1 to about 1:3, or about 1:1 to about 1:32, including all ranges and subranges thereof.

In a further aspect, the oral care compositions of the disclosure may comprise an anionic surfactant in an amount from about 0.5 to about 20 wt. %, based on the total weight of the oral care composition. For example, the amount of anionic surfactant present in the oral care composition may be from about 0.5 to about 20 wt. %, about 0.5 to about 15 wt. %, about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; about 1 to about 20 wt. %, about 1 to about 15 wt. %, about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; about 2 to about 20 wt. %, about 2 to about 15 wt. %, about 2 to about 10 wt. %, about 2 to about 8 wt. %, about 2 to about 6 wt. %, about 2 to about 5 wt. %, about 2 to about 4 wt. %, or about 2 to about 3 wt. %, including ranges and subranges thereof, based on the total weight of the oral care composition.

The anionic surfactants may be selected from water-soluble, water-miscible salts of alkyl sulfate, such as those having from 8 to 20 carbon atoms in the alkyl radical (e.g., sodium alkyl sulfate) and the water-soluble or water-miscible salts of sulfonated monoglycerides of fatty acids having from 8 to 20 carbon atoms. Examples of suitable anionic surfactants include sodium lauryl sulfate, sodium lauryl sarcosinate, sodium cocoyl methyl taurate, sodium monoglyceride sulfate, sodium cetaryl sulfate, potassium cocoyl glycinate, sodium lauryl phosphate, sodium lauryl lactylate, sodium lauryl sulfoacetate, sodium lauryl glutamate, sodium lauryl isethionate, sodium laureth carboxylate, sodium dodecyl benzenesulfonate, and combinations thereof. In one embodiment, sodium lauryl sulfate is a preferred swelling surfactant. Additional anionic surfactants are disclosed in U.S. Pat. No. 3,959,458, which is incorporated herein in its entirety for all purposes.

Further anionic surfactants of mention include long chain alkyl (C₆-C₂₂) materials, such as long chain alkyl sulfates, long chain alkyl sulfonates, long chain alkyl phosphates, long chain alkyl ether sulfates, long chain alkyl alpha olefin sulfonates, long chain alkyl taurates, long chain alkyl isethionates (SCI), long chain alkyl glyceryl ether sulfonates (AGES), sulfosuccinates and the like. These anionic surfactants can be alkoxylated, for example, ethoxylated, although alkoxylation is not required.

In still another aspect, the oral care compositions of the disclosure can comprise an anti-tarter agent(s), e.g., in an amount ranging from about 10 to about 40 wt. %, based on the total weight of the oral care composition. For example, the oral care compositions may include an anti-tarter agent in an amount from about 10 to about 40 wt. %, about 10 to about 35 wt. %, about 10 to about 30 wt. %, about 10 to about 28 wt. %, about 10 to about 26 wt. %, about 10 to about 24 wt. %, about 10 to about 22 wt. %; about 12 to about 40 wt. %, about 12 to about 35 wt. %, about 12 to about 30 wt. %, about 12 to about 28 wt. %, about 12 to about 26 wt. %, about 12 to about 24 wt. %, about 12 to about 22 wt. %; about 14 to about 40 wt. %, about 14 to about 35 wt. %, about 14 to about 30 wt. %, about 14 to about 28 wt. %, about 14 to about 26 wt. %, about 14 to about 24 wt. %, about 14 to about 22 wt. %; about 16 to about 40 wt. %, about 16 to about 35 wt. %, about 16 to about 30 wt. %, about 16 to about 28 wt. %, about 16 to about 26 wt. %, about 16 to about 24 wt. %, about 16 to about 22 wt. %, including all ranges and subranges therebetween, based on the total weight of the oral care composition.

In some aspects, the oral care composition of the disclosure may include anti-tarter agent(s) that have anti-calculus properties. In some instances, the anti-tarter agents are chosen from alkali-metal pyrophosphates, hypophosphite-containing polymers, organic phosphocitrates, phosphocitrates, polyphosphates, and combinations of two or more thereof. The anti-tarter agents may be a pyrophosphate chosen from dialkali or tetra-alkali metal pyrophosphate salts, such as Na₄P₂O₇ (TSPP), K₄P₂O₀₇, Na₂K₂P₂O₇, Na₂K₂H₂O₇, and K₂H₂P₂O₀₇, long chain polyphosphates such as sodium hexametaphosphate, and cyclic phosphates such as sodium trimetaphosphate.

In a further aspect, the oral care compositions of the disclosure can include a fluoride ion source in an amount from about 0.1 to about 4 wt. %, based on the total weight of the oral care composition. In some cases, the amount of fluoride ion source present in the oral care composition may be from about 0.1 to about 4 wt. %, about 0.1 to about 3 wt. %, about 0.1 to about 2 wt. %, about 0.1 to about 1.5 wt. %, about 0.1 to about 1 wt. %; about 0.5 to about 4 wt. %, about 0.5 to about 3 wt. %, about 0.5 to about 2 wt. %, about 0.5 to about 1.5 wt. %, about 0.5 to about 1 wt. %; about 0.75 to about 4 wt. %, about 0.75 to about 3 wt. %, about 0.75 to about 2 wt. %, about 0.75 to about 1.5 wt. %, about 0.75 to about 1 wt. %, including ranges and subranges therebetween, based on the weight of the oral care composition. Additionally or alternatively, the fluoride ion source may be incorporated into the oral care composition in an amount that provides between 100 and 3000 ppm, preferably between 500 and 2000 ppm of fluoride ions.

The fluoride ion source may be a fluorine-containing compound. Suitable ionic fluorine-containing compounds include fluoride salts, such as amine fluorides, alkali metal fluoride salts (e.g., sodium fluoride), and monofluorophosphate salts, such as alkali metal monofluorophosphate salts (e.g., sodium monofluorophosphate). In some embodiments, the fluoride ion source may be chosen from fluoride salts, such as sodium fluoride, potassium fluoride, calcium fluoride, zinc fluoride, stannous fluoride, zinc ammonium fluoride, sodium monofluorophosphate, potassium monofluorophosphate, laurylamine hydrofluoride, and combinations of two or more thereof. In at least one embodiment, the fluoride ion source may comprise or consist of sodium monofluorophosphate.

In some aspects, the oral care compositions of the disclosure may include one or more abrasive(s) in an amount of about 1 to about 30 wt. %, based on the total weight of the oral care composition. For example, the oral care composition may include an abrasive in an amount from about 1 to about 30 wt. %, about 1 to about 27.5 wt. %, about 1 to about 25 wt. %, about 1 to about 22.5 wt. %, about 1 to about 20 wt. %, about 1 to about 18 wt. %, about 1 to about 16 wt. %; about 5 to about 30 wt. %, about 5 to about 27.5 wt. %, about 5 to about 25 wt. %, about 5 to about 22.5 wt. %, about 5 to about 20 wt. %, about 5 to about 18 wt. %, about 5 to about 16 wt. %; about 10 to about 30 wt. %, about 10 to about 27.5 wt. %, about 10 to about 25 wt. %, about 10 to about 22.5 wt. %, about 10 to about 20 wt. %, about 10 to about 18 wt. %, about 10 to about 16 wt. %; about 15 to about 30 wt. %, about 15 to about 27.5 wt. %, about 15 to about 25 wt. %, about 15 to about 22.5 wt. %, about 15 to about 20 wt. %, or about 15 to about 18 wt. %, based on the total weight of the oral care composition.

The abrasives may be chosen from silica (participated silica and/or fused silica) (e.g., wherein the silica is not a high cleaning silica), zinc orthophosphate, plastics particles, alumina, hydrated alumina, and calcium pyrophosphate or mixtures thereof. Suitable abrasives that may be included in the oral care composition include, but are not limited to, silica, alumina, insoluble phosphates, calcium carbonate, resinous abrasives, and a combination of two or more thereof. In at least one embodiment, the oral care composition does not include fused silica. In at least one other embodiment, the oral care composition includes fused silica.

In still a further aspect, the oral care composition of the disclosure may include a buffering system. The buffering system may include one or more buffering agents. While in some instances, the buffering system comprises pH adjusters, in other instances the buffering system only consists of buffering agents. The oral care composition may, additionally or alternatively, comprise one or more pH adjusters to increase or decrease the overall pH of the oral care composition. For example, one or more acids may be included to decrease the pH of the oral care composition. Examples of suitable acids for decreasing the pH of the oral care composition include, but are not limited to, citric acid, acetic acid, and the like. The oral care composition may include one or more bases, such as sodium hydroxide, potassium hydroxide and the like, to increase the pH of the oral care composition. Additional or alternative acids and bases that are suitable for adjusting the pH of the oral care composition are readily known to one of ordinary skill in the art.

The amount of the pH adjuster in the oral care composition may be based on the desired pH of the final oral care composition and/or product. For example, the total amount of the pH adjuster may range from about 0.05 to about 20 wt. %, based on the total weight of the oral care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the oral care composition.

As noted above, the oral care compositions of the disclosure may have a pH from 4.5 to about 10, 4.5 to about 9, 4.5 to about 8, 4.5 to about 7, 4.5 to about 6; about 5 to about 10, about 5 to about 9, about 5 to about 8, about 5 to about 7, about 5 to about 6; about 6 to about 10, about 6 to about 9, about 6 to about 8, about 6 to about 7; about 7 to about 10, about 7 to about 9, or about 7 to about 8, including any ranges and subranges therebetween. In some cases, the buffering system is present in the oral care composition in an amount effective to provide a pH greater than about 4.5, optionally greater than about 5.0, optionally greater than about 5.5, optionally greater than about 6.0, optionally greater than about 6.5, optionally greater than about 7.0, optionally greater than about 7.5, optionally greater than about 8.0, optionally greater than about 8.5 or up to about 9.0.

The oral care compositions may include any of the following additional ingredients in an amount of from about 0.01 to about 15 wt. %, based on the total weight of the oral care composition. In some instances, the amount of additional ingredients present in the oral care composition is from about 0.01 to about 12.5 wt. %, about 0.01 to about 10 wt. %, about 0.01 to about 8 wt. %, about 0.01 to about 6 wt. %, about 0.01 to about 4 wt. %, about 0.01 to about 3 wt. %, about 0.01 to about 2 wt. %, about 0.01 to about 1 wt. %, about 0.01 to about 0.5 wt. %, about 0.01 to about 0.1 wt. %; about 0.1 to about 12.5 wt. %, about 0.1 to about 10 wt. %, about 0.1 to about 8 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.5 wt. %, about 0.1 to about 0.1 wt. %; about 0.5 to about 12.5 wt. %, about 0.5 to about 10 wt. %, about 0.1 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; about 0.75 to about 12.5 wt. %, about 0.75 to about 10 wt. %, about 0.75 to about 8 wt. %, about 0.75 to about 6 wt. %, about 0.75 to about 5 wt. %, about 0.75 to about 4 wt. %, about 0.75 to about 3 wt. %, about 0.75 to about 2 wt. %, about 0.75 to about 1 wt. %; about 1 to about 12.5 wt. %, about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; about 2 to about 5 wt. %, about 2 to about 4 wt. %, about 2 to about 3 wt. %; about 3 to about 12.5 wt. %, about 3 to about 10 wt. %, about 3 to about 8 wt. %, about 3 to about 6 wt. %, about 3 to about 5 wt. %, or about 3 to about 4 wt. %, including any range or subrange therebetween, based on the total weight of the oral care composition.

In yet another aspect, the oral care compositions of the disclosure may comprise one or more additional ingredients including, e.g., non-hydrogen peroxide whitening agents, nonionic surfactants, amphoteric surfactants, cationic surfactants, stannous salts and/or ions thereof, thickening agents, preservatives, emulsify, colorants, pigments, flavoring agents, sweeteners, or the like.

In some embodiments, the oral care composition may comprise a nonionic surfactant. Nonionic surfactants useful herein may include those compounds produced by the condensation of alkylene oxide groups (hydrophilic in nature) with an organic hydrophobic compound, which may be aliphatic or alkyl-aromatic in nature. Non-limiting examples of suitable nonionic surfactants include polyoxyethylene sorbitan esters (sold under the trade name Tweens), polyoxyl 40 hydrogenated castor oil, fatty alcohol ethoxylates, ethylene oxide condensates of aliphatic alcohols, long chain tertiary amine oxides, long chain tertiary phosphaine oxides, lauryl glucoside (sold under the trade name Plantaren 1200 UP) and long chain dialkyl sulfoxides. Suitable nonionic surfactants with a HLB of 7 or more include sucrose laurate, sucrose cocoate, sucrose stearate; Steareth 20, 21, or 100, and PEG 20 Sorbitan Monostearate (commercially available as Tween 60). In at least one embodiment, the nonionic surfactants are chosen from polyethoxylated sorbitol esters, in particular polyethoxylated sorbitol monoesters; polycondensates of ethylene oxide and propylene oxide (poloxamers), for instance the products marketed under the trade name PLURONIC by BASF-Wyandotte; condensates of propylene glycol; polyethoxylated hydrogenated castor oil, for instance, cremophors; and sorbitan fatty esters.

Non-limiting examples of amphoteric surfactants include, for example, long chain imidazoline derivatives such as the product marketed under the trade name ‘Miranol C2M’ by Miranol; long chain alkyl betaines, such as the product marketed under the tradename ‘Empigen BB’ by Albright+Wilson, and long chain alkyl amidoalkyl betaines, such as cocamidopropylbetaine, and mixtures thereof.

In still a further aspect, the oral care composition of the disclosure may comprise amphoteric surfactants chosen from derivatives of aliphatic secondary and tertiary amines, in which the aliphatic radical can be a straight chain or branched and wherein one of the aliphatic substituents contains from about 8 to about 18 carbon atoms and one contains an anionic water-solubilizing group, such as carboxylate, sulfonate, sulfate, phosphate, or phosphonate. Other exemplary amphoteric surfactants are betaines, such as cocamidopropyl betaine, lauryl dimethyl betaine (sold under the trade name Macat LB), cetyl dimethyl betaine, and cocoamphodiacetate. Additional amphoteric surfactants and nonionic surfactants can be found in U.S. Pat. No. 4,051,234, which is incorporated herein in its entirety for all purposes.

Examples of cationic surfactants that may be present in the oral care composition include cetyl pyridinium chloride, coamidopropyl PG dimonium chloride phosphate (Phospholipid CDM), myristylamidopropyl PG dimonium chloride phosphate (Phospholipid PTM), stearamidopropyl PG dimonium chloride phosphate (Phospholipid SV), steapyrium chloride (Catemol WPC), and other suitable cationic materials. The cationic surfactants may be D,L-2-pyrrolidone-5-carbo-xylic acid salt of ethyl-N-cocoyl-L-arginate, marketed under the trade name CAE by Ajinomoto Co. Inc.

The oral care composition may further comprise one or more colorants. The colorants may be a pigment, a dye, or mixtures thereof. Non-limiting examples of pigments include titanium dioxide, zinc oxide, kaolin, mica etc. Non-limiting examples of dyes include food dyes suitable for food, drug and cosmetic applications, and mixtures thereof. Some color agents (colorants) are known as FD&C dyes. In some embodiments, the colorants may be present in an amount ranging from about 0.0001% wt. % to about 0.4% wt. %, including all percentages and subranges therebetween, based on the total weight of the oral care composition. In further embodiments, the colorants may be present in an amount ranging from about 0.0001% wt. % to about 4% wt. %, including all percentages and subranges therebetween, based on the total weight of the oral care composition.

Examples of flavoring agents (flavors and/or flavoring materials) include: menthol; carvone; anethole; methyl salicylate; and the oils of spearmint, peppermint, wintergreen, sassafras, clove, sage, eucalyptus, marjoram, cinnamon, lemon, lime, grapefruit, kumquat, tangerine, and orange. Examples of sweeteners (sweetening agents) include sucrose, lactose, maltose, sorbitol, xylitol, sodium cyclamate, perillartine, L-aspartyl-L-phenylalanine methyl ester (aspartame), and saccharine.

In some aspects, the oral care compositions of the disclosure may include additional and/or optional thickeners other than polyvinyl pyrrolidone. Illustrative additional or optional thickeners other than polyvinyl pyrrolidone may be or include, but are not limited to, carbomers (e.g., carboxyvinyl polymers), carrageenans (e.g., Irish moss, carrageenan, iota-carrageenan, etc.), high molecular weight polyethylene glycols (e.g., CARBOWAX®, which is commercially available from The Dow Chemical Company of Midland, Mich.), cellulosic polymers, hydroxyethylcellulose, carboxymethylcellulose, and salts thereof (e.g., CMC sodium), natural gums (e.g., karaya, xanthan, gum arabic, and tragacanth), colloidal magnesium aluminum silicate, and the like, and mixtures or combinations of two or more thereof. In one embodiment, the oral care composition includes a thickening system comprising a polymer selected from polyvinyl pyrrolidone, a polyacrylate, a polymethacrylate, a polyitaconate, an acrylamide, 2-acrylamido-2-methylpropane sulfonic acid (AMPS); and a combination of two or more thereof.

In some aspects, the oral care composition may optionally include a whitening agent other than hydrogen peroxide. The whitening agent may be substantially anhydrous oxygen generating compounds. For example, the additional whitening agent may be a peroxide other than hydrogen peroxide, a metal chlorite, and/or a persulfate. Examples of peroxide phases include hydroperoxides, peroxides of alkali and alkaline earth metals, organic peroxy compounds, peroxy acids, pharmaceutically-acceptable salts thereof, and combinations of two or more thereof. Peroxides of alkali and alkaline earth metals include lithium peroxide, potassium peroxide, sodium peroxide, magnesium peroxide, calcium peroxide, barium peroxide, and a combination of two or more thereof. Organic peroxy compounds include urea peroxide, glyceryl hydrogen peroxide, alkyl hydrogen peroxides, dialkyl peroxides, alkyl peroxy acids, peroxy esters, diacyl peroxides, benzoyl peroxide, and monoperoxyphthalate, and a combination of two or more thereof. Peroxy acids and their salts include organic peroxy acids such as alkyl peroxy acids, and monoperoxyphthalate and a combination of two or more thereof, as well as inorganic peroxy acid salts such as and perborate salts of alkali and alkaline earth metals such as lithium, potassium, sodium, magnesium, calcium and barium, and a mixture of two or more thereof. Non-limiting examples of metal chlorites include calcium chlorite, barium chlorite, magnesium chlorite, lithium chlorite, sodium chlorite, and potassium chlorite.

In other aspects, the oral care composition of the disclosure may comprises any of the following materials in any desired amount to achieve a desired effect in the composition: one or more alkaline salts, for example, sodium chloride, sodium sulfate, sodium carbonate, sodium bicarbonate and/or their equivalents; sequestrants, for example, tetrasodium EDTA, and/or their equivalents; biocides, for example, Triclosan (2,4,4′-trichloro-2′-hydroxydiphenyl ether), DMDM hydantoin, formaldehyde and/or imidazolidinyl urea, and/or their equivalents; organic acids, for example, citric acid and/or formic acid and/or their equivalents; viscosity modifiers; preservatives, for example, phenoxyethanol, formaldehyde solution, parabens, pentanediol or sorbic acid; pearlizing agents, for example, glycol distearic esters, such as ethylene glycol distearate, but also fatty acid monoglycol esters; stabilizers, for example, metal salts of fatty acids, such as e.g. magnesium stearate, aluminum stearate and/or zinc stearate; and dyes and pigments that are approved and suitable for oral care compositions.

In some aspects, the oral care compositions of the disclosure do not include any high cleaning silica. Further, in some aspects, the oral care composition of the disclosure does not include any high cleaning silica and demonstrates increased hydrogen peroxide stability (e.g., as measured by active oxygen levels) relative to a control formulation that comprises high cleaning silica.

In a further aspect, the oral care composition can comprise the following:
a) Hydrogen peroxide solution (e.g., from 0.1%-10% by wt. of the solution) (e.g., from 0.05%-5% by wt. of hydrogen peroxide relative to the total weight of the oral care composition and from 0.05%-5% by wt. of water relative to the total weight of the oral care composition);
b) Calcium pyrophosphate (e.g., from 5%-30% by wt.); and
c) Polyvinylpyrrolidone (e.g., from 1%-15% by wt.).

In a further aspect, the oral care composition can comprise the following:
a) Hydrogen peroxide solution (e.g., from 0.05%-5% by wt. of hydrogen peroxide relative to the total weight of the oral care composition and from 0.05%-5% by wt. of water relative to the total weight of the oral care composition);
b) Calcium pyrophosphate (e.g., from 5%-30% by wt.); and
c) Polyvinylpyrrolidone (e.g., from 1%-15% by wt.);
wherein the hydrogen peroxide and polyvinylpyrrolidone are not bound or complexed together prior to being added together to the composition (e.g., wherein they are not added as a preformed complex).

In a further aspect, the oral care composition can comprise the following:
a) Hydrogen peroxide solution (e.g., from 0.1%-10% by wt. of the solution) (e.g., from 0.05%-5% by wt. of hydrogen peroxide relative to the total weight of the oral care composition and from 0.05%-5% by wt. of water relative to the total weight of the oral care composition));
b) Calcium pyrophosphate (e.g., from 5%-30% by wt.);
c) Polyvinylpyrrolidone (e.g., from 1%-15% by wt.); and
wherein the composition does not contain any high cleaning silica.

In a further aspect, the oral care composition can comprise the following:
a) Hydrogen peroxide solution (e.g., from 0.1%-10% by wt. of the solution) (e.g., from 0.05%-5% by wt. of hydrogen peroxide relative to the total weight of the oral care composition and from 0.05%-5% by wt. of water relative to the total weight of the oral care composition);
b) Calcium pyrophosphate (e.g., from 5%-30% by wt.);
c) Polyvinylpyrrolidone (e.g., from 1%-15% by wt.);
d) Tetrasodium pyrophosphate (e.g., from 0.5%-4% by wt.);
e) Sodium acid pyrophosphate;
f) Silicon dioxide (e.g., fumed silica) (e.g., from 1-10% by wt.) (e.g., from 2-5% by wt.); and
wherein the hydrogen peroxide and polyvinylpyrrolidone are not bound or complexed together prior to being added together to the composition (e.g., wherein they are not added as a preformed complex).

In still a further aspect, the oral care composition can comprise the following:
a) Hydrogen peroxide solution (e.g., from 0.1%-10% by wt. of the solution) (e.g., from 0.05%-5% by wt. of hydrogen peroxide relative to the total weight of the oral care composition and from 0.05%-5% by wt. of water relative to the total weight of the oral care composition);
b) Calcium pyrophosphate (e.g., from 5%-30% by wt.);
c) Polyvinylpyrrolidone (e.g., from 1%-15% by wt.);
d) Tetrasodium pyrophosphate (e.g., from 0.5%-4% by wt.);
e) Sodium acid pyrophosphate (e.g., from 0.1%-1% by wt.);
f) Silicon dioxide (e.g., fumed silica) (e.g., from 1-10% by wt.) (e.g., from 2-5% by wt.); and
wherein the hydrogen peroxide and polyvinylpyrrolidone are not bound or complexed together prior to being added together to the composition (e.g., wherein they are not added as a preformed complex).

In still a further aspect, the oral care composition can comprise the following:
a) Hydrogen peroxide solution (e.g., from 0.1%-10% by wt. of the solution) (e.g., from 0.05%-5% by wt. of hydrogen peroxide relative to the total weight of the oral care composition and from 0.05%-5% by wt. of water relative to the total weight of the oral care composition);
b) Calcium pyrophosphate (e.g., from 5%-30% by wt.);
c) Polyvinylpyrrolidone (e.g., from 5%-15% by wt.);
d) Tetrasodium pyrophosphate (e.g., from 0.5%-4% by wt.);
e) Sodium acid pyrophosphate (e.g., from 0.1%-2% by wt.);
f) Propylene glycol (e.g., from 25%-55% by wt.);
g) Glycerin (e.g., from 5%-15% by wt.);
h) Silicon dioxide (e.g., fumed silica) (e.g., from 1-10% by wt.) (e.g., from 2-5% by wt.); and
wherein the hydrogen peroxide and polyvinylpyrrolidone are not bound or complexed together prior to being added together to the composition and wherein the composition does not contain any high cleaning silica.

In yet further aspect, the oral care composition can comprise the following:
a) Hydrogen peroxide solution (e.g., from 0.1%-10% by wt. of the solution) (e.g., from 0.05%-5% by wt. of hydrogen peroxide relative to the total weight of the oral care composition and from 0.05%-5% by wt. of water relative to the total weight of the oral care composition);
b) Calcium pyrophosphate (e.g., from 5%-30% by wt.);
c) Polyvinylpyrrolidone (e.g., from 1%-15% by wt.);
d) Tetrasodium pyrophosphate (e.g., from 0.5%-4% by wt.);
e) Sodium acid pyrophosphate (e.g., from 0.1%-1% by wt.); and
f) Propylene glycol;
g) Polyethylene Glycol/Polypropylene Glycol Copolymer (e.g., block copolymer);
h) Silicon dioxide (e.g., fumed silica) (e.g., from 1-10% by wt.) (e.g., from 2-5% by wt.); and
i) Glycerin.

In yet further aspect, the oral care composition can comprise the following:
a) Hydrogen peroxide solution (e.g., from 0.1%-10% by wt. of the solution) (e.g., from 0.05%-5% by wt. of hydrogen peroxide relative to the total weight of the oral care composition and from 0.05%-5% by wt. of water relative to the total weight of the oral care composition);
b) Calcium pyrophosphate (e.g., from 5%-30% by wt.);
c) Polyvinylpyrrolidone (e.g., from 1%-15% by wt.);
d) Tetrasodium pyrophosphate (e.g., from 0.5%-4% by wt.);
e) Sodium acid pyrophosphate (e.g., from 0.1%-1% by wt.);
f) Propylene glycol
g) Glycerin;
h) Silicon dioxide (e.g., fumed silica) (e.g., from 1-10% by wt.) (e.g., from 2-5% by wt.); and
wherein the hydrogen peroxide and polyvinylpyrrolidone are not bound or complexed together prior to being added together to the composition (e.g., wherein they are not added as a preformed complex) and wherein the composition does not contain any high cleaning silica.

In yet another aspect, the oral care composition(s) can comprise the following:
a) from about 35 wt. % to about 85 wt. % of an anhydrous solvent system (e.g., 35 wt. % to about 75 wt. %);
b) from about 0.02 wt. % to about 30 wt. % of a peroxide solution comprising:
   (i) from about 0.01 wt. % to about 15 wt. % of hydrogen peroxide, and
   (ii) from about 0.01 wt. % to about 15 wt. % of water;
c) from about 1 wt. % to about 20 wt. % of a thickening system;
d) wherein the oral care composition has a total water content of less than about 30 wt. %;
wherein the composition does not contain any high cleaning silica; and wherein all weight percentages are based on the total weight of the oral care composition.

In a further aspect, the oral care composition can comprise the following:

a) Hydrogen peroxide solution (e.g., from 0.1%-10% by wt. of the solution) (e.g., from 0.05%-5% by wt. of hydrogen peroxide relative to the total weight of the oral care composition and from 0.05%-5% by wt. of water relative to the total weight of the oral care composition);
b) Calcium pyrophosphate (e.g., from 5%-30% by wt.);
c) Polyvinylpyrrolidone (e.g., from 1%-15% by wt.);
d) Silicon dioxide (e.g., fumed silica) (e.g., from 1-10% by wt.) (e.g., from 2-5% by wt.); and
wherein the composition does not contain any high cleaning silica and wherein the hydrogen peroxide and polyvinylpyrrolidone are not bound or complexed together prior to being added together in the composition.

In a further aspect, the oral care composition can comprise the following:
a) Hydrogen peroxide solution (e.g., from 0.1%-10% by wt. of the solution) (e.g., from 0.05%-5% by wt. of hydrogen peroxide relative to the total weight of the oral care composition and from 0.05%-5% by wt. of water relative to the total weight of the oral care composition);
b) Calcium pyrophosphate (e.g., from 5%-30% by wt.);
c) Polyvinylpyrrolidone (e.g., from 1%-15% by wt.);
d) Silicon dioxide (e.g., fumed silica); and
wherein the composition does not contain any high cleaning silica and wherein the hydrogen peroxide and polyvinylpyrrolidone are not bound or complexed together prior to being added together in the composition.

In a further aspect, the oral care composition can comprise the following:
a) Hydrogen peroxide solution (e.g., from 0.1%-10% by wt. of the solution) (e.g., from 0.05%-5% by wt. of hydrogen peroxide relative to the total weight of the oral care composition and from 0.05%-5% by wt. of water relative to the total weight of the oral care composition);
b) Calcium pyrophosphate (e.g., from 5%-30% by wt.);
c) Polyvinylpyrrolidone (e.g., from 1%-15% by wt.);
d) a PEG/PPG Copolymer (e.g., block copolymer) (e.g., from 5%-15% by wt.);
e) Propylene glycol;
f) Silicon dioxide (e.g., fumed silica) (e.g., from 1-10% by wt.) (e.g., from 2-5% by wt.); and
wherein the composition does not contain any high cleaning silica and wherein the hydrogen peroxide and polyvinylpyrrolidone are not bound or complexed together prior to being added together in the composition.

In a further aspect, the oral care composition can comprise the following:
a) Hydrogen peroxide solution (e.g., from 0.1%-10% by wt. of the solution) (e.g., from 0.05%-5% by wt. of hydrogen peroxide relative to the total weight of the oral care composition and from 0.05%-5% by wt. of water relative to the total weight of the oral care composition);
b) Calcium pyrophosphate (e.g., from 5%-30% by wt.);
c) Polyvinylpyrrolidone (e.g., from 1%-15% by wt.);
d) Silicon dioxide (e.g., fumed silica);
e) Propylene glycol;
f) Polyethylene Glycol/Polypropylene Glycol Copolymer (e.g., block copolymer); and
wherein the composition does not contain any high cleaning silica and wherein the hydrogen peroxide and polyvinylpyrrolidone are not bound or complexed together prior to being added together in the composition.

In a further aspect, the oral care composition can comprise the following:
a) Hydrogen peroxide solution (e.g., from 0.1%-10% by wt. of the solution) (e.g., from 0.05%-5% by wt. of hydrogen peroxide relative to the total weight of the oral care composition and from 0.05%-5% by wt. of water relative to the total weight of the oral care composition);
b) Calcium pyrophosphate (e.g., from 5%-30% by wt.);
c) Polyvinylpyrrolidone (e.g., from 1%-15% by wt.);
d) Propylene glycol;
e) Polyethylene Glycol/Polypropylene Glycol Copolymer (e.g., block copolymer);
f) Tetrasodium pyrophosphate (e.g., from 0.5%-4% by wt.);
g) Sodium acid pyrophosphate (e.g., from 0.1%-1% by wt.); and
Wherein the composition does not contain any high cleaning silica and wherein the hydrogen peroxide and polyvinylpyrrolidone are not bound or complexed together prior to being added together in the composition.

In a further aspect, the oral care composition can comprise the following:
a) Hydrogen peroxide solution (e.g., from 0.1%-10% of the solution) (e.g., from 0.05%-5% by wt. of hydrogen peroxide relative to the total weight of the oral care composition and from 0.05%-5% by wt. of water relative to the total weight of the oral care composition);
b) Calcium pyrophosphate;
c) Polyvinylpyrrolidone;
d) Tetrasodium pyrophosphate;
e) Sodium acid pyrophosphate;
f) Propylene glycol;
g) Glycerin; and
Wherein the composition does not contain any high cleaning silica and wherein the hydrogen peroxide and polyvinylpyrrolidone are not bound or complexed together prior to being added together in the composition.

In a further aspect, the oral care composition of the disclosure can comprise the following:
a) Hydrogen peroxide from 0.05%-7% by wt. relative to the total weight of the oral care composition;
b) Water from 0.05%-7% by wt. relative to the total weight of the oral care composition;
c) Calcium pyrophosphate (e.g., from 5%-30% by wt.);
d) Propylene glycol;
e) Polyethylene Glycol/Polypropylene Glycol Copolymer (e.g., block copolymer) (e.g., 116/66 Copolymer); and
f) Polyvinylpyrrolidone (e.g., from 1%-15% by wt.)
wherein the hydrogen peroxide and polyvinylpyrrolidone are not bound or complexed together prior to being added together to the composition (e.g., wherein they are not added as a preformed complex).

In a further aspect, the oral care composition of the disclosure can comprise the following:

a) Hydrogen peroxide from 0.05%-7% by wt. relative to the total weight of the oral care composition (e.g., about 0.1%, about 1%, about 2%, about 3%, or about 4% by wt. of hydrogen peroxide);
b) Water from 0.05%-7% by wt. relative to the total weight of the oral care composition;
c) Calcium pyrophosphate (e.g., from 5%-30% by wt.);
d) Polyvinylpyrrolidone (e.g., from 1%-15% by wt.);
e) Propylene glycol;
f) Polyethylene Glycol/Polypropylene Glycol Copolymer (e.g., block copolymer) (e.g., 116/66 Copolymer); and
wherein the composition does not contain any high cleaning silica and wherein the hydrogen peroxide and polyvinylpyrrolidone are not bound or complexed together prior to being added together in the composition (e.g., wherein they are not added as a preformed complex).

In a further aspect, the oral care composition of the disclosure can comprise the following:
a) Hydrogen peroxide from 0.05%-7% by wt. relative to the total weight of the oral care composition;
b) Water from 0.05%-7% by wt. relative to the total weight of the oral care composition;
c) Calcium pyrophosphate (e.g., from 5%-30% by wt.);
d) Polyvinylpyrrolidone (e.g., from 1%-15% by wt.);
e) Silicon dioxide (e.g., fumed silica);
f) Propylene glycol;
g) Polyethylene Glycol/Polypropylene Glycol Copolymer (e.g., block copolymer) (e.g., 116/66 Copolymer); and
wherein the composition does not contain any high cleaning silica and wherein the hydrogen peroxide and polyvinylpyrrolidone are not bound or complexed together prior to being added together in the composition (e.g., wherein they are not added as a preformed complex).

In still a further aspect, the oral care composition of the disclosure comprises:
from about 15 wt. % to about 90 wt. % of an anhydrous solvent system (e.g., wherein the system comprises propylene glycol and a polyethylene glycol/polypropylene glycol copolymer (e.g., block copolymer) (e.g., 116/66 copolymer));
liquid peroxide source in an amount effective to provide from about 0.01 wt. % to about 15 wt. % of hydrogen peroxide (e.g., about 0.1%, about 1%, about 2%, about 3%, or about 4% hydrogen peroxide by wt.);
a total water content of less than 35 wt. % (e.g., from 0.1%-15% by wt.); and from about 1 wt. % to about 20 wt. % of a thickening system;
wherein the weight ratio of total water content to polymer is from about 1:1 to about 1:20; wherein the oral care composition has a pH of 4.5 to about 9.0;
wherein the oral care composition is a toothpaste; and
wherein all weight percentages are based on the total weight of the oral care composition.

In still a further aspect, the oral care composition of the disclosure comprises:
from about 15 wt. % to about 90 wt. % of an anhydrous solvent system (e.g., wherein the anhydrous solvent system comprises a solvent selected from: a block copolymer of ethylene oxide and propylene oxide; ethylene glycol; propylene glycol; butylene glycol; hexylene glycol; pentylene glycol; 1,3-propanediol; diethylene glycol; polyethylene glycol; a polyethylene glycol/polypropylene glycol copolymer; dipropylene glycol; caprylyl glycol; glycerin; and a combination of two or more thereof) (e.g., wherein the system comprises propylene glycol and a polyethylene glycol/polypropylene glycol copolymer (e.g., block copolymer) (e.g., 116/66 Copolymer);
liquid peroxide source in an amount effective to provide from about 0.01 wt. % to about 15 wt. % of hydrogen peroxide (e.g., about 0.1%, about 1%, about 2%, about 3%, or about 4% hydrogen peroxide by wt.);
a total water content of less than 35 wt. % (e.g., from 0.1%-15% by wt.); and from about 1 wt. % to about 20 wt. % of a thickening system;
wherein the weight ratio of total water content to polymer is from about 1:1 to about 1:20;
wherein the oral care composition has a pH of 4.5 to about 9.0;
wherein the oral care composition is a toothpaste; and
wherein the composition does not contain any high cleaning silica and/or wherein the hydrogen peroxide and polyvinylpyrrolidone are not bound or complexed together prior to being added together in the composition (e.g., wherein they are not added as a preformed complex);
wherein all weight percentages are based on the total weight of the oral care composition

In yet another aspect, the oral care composition of the disclosure can comprise:
From about 15 wt. % to about 90 wt. % of an anhydrous solvent system (e.g., wherein the anhydrous solvent system comprises a solvent selected from: a block copolymer of ethylene oxide and propylene oxide; ethylene glycol; propylene glycol; butylene glycol; hexylene glycol; pentylene glycol; 1,3-propanediol; diethylene glycol; polyethylene glycol; a polyethylene glycol/polypropylene glycol copolymer; dipropylene glycol; caprylyl glycol; glycerin; and a combination of two or more thereof) (e.g., wherein the system comprises propylene glycol and a polyethylene glycol/polypropylene glycol copolymer (e.g., block copolymer) (e.g., 116/66 Copolymer);
from about 0.01 wt. % to about 15 wt. % of a peroxide source (e.g., about 0.1%, about 1%, about 2%, about 3%, or about 4% hydrogen peroxide by wt.); and a total water content of less than about 30 wt. % (e.g., from 0.1%-15% by wt.);
wherein the peroxide source is provided in a liquid form;
wherein the oral care composition has a pH of from about 4.5 to about 9.0; and
wherein all weight percentages are based on the total weight of the oral care composition.

In yet another aspect, the oral care composition of the disclosure can comprise:
From about 15 wt. % to about 90 wt. % of an anhydrous solvent system (e.g., wherein the anhydrous solvent system comprises a solvent selected from: a block copolymer of ethylene oxide and propylene oxide; ethylene glycol; propylene glycol; butylene glycol; hexylene glycol; pentylene glycol; 1,3-propanediol; diethylene glycol; polyethylene glycol; a polyethylene glycol/polypropylene glycol copolymer; dipropylene glycol; caprylyl glycol; glycerin; and a combination of two or more thereof) (e.g., wherein the system comprises propylene glycol and a polyethylene glycol/polypropylene glycol copolymer (e.g., block copolymer) (e.g., 116/66 Copolymer);
from about 0.01 wt. % to about 15 wt. % of a peroxide source (e.g., about 0.1%, about 1%, about 2%, about 3%, or about 4% hydrogen peroxide by wt.); and a total water content of less than about 30 wt. % (e.g., from 0.1%-15% by wt.);
wherein the peroxide source is provided in a liquid form;
wherein the oral care composition has a pH of from about 4.5 to about 9.0;
wherein the composition does not contain any high cleaning silica and/or wherein the hydrogen peroxide and polyvinylpyrrolidone are not bound or complexed together prior to being added together in the composition (e.g., wherein they are not added as a preformed complex); and
wherein all weight percentages are based on the total weight of the oral care composition.

In further aspects, any of the oral care compositions disclosed herein can comprise a liquid hydrogen peroxide solution (e.g., from 0.05%-7.5% hydrogen peroxide based on the wt. of the total composition) and polyvinylpyrrolidone (PVP). In some aspects, where the oral care composition comprises liquid hydrogen peroxide solution and PVP, the PVP and HP are not complexed or bound when added to the oral care composition and may form a complex in situ after the addition to the oral care composition (e.g., a toothpaste). In this aspect, the PVP and HP are not bound or complexed together prior to their addition to the oral care composition (e.g., they are not added as a preformed complex).

Aspects of the invention are directed to methods for manufacturing and using oral care compositions discussed herein. In some embodiments, the methods include producing the oral care compositions using a hydrogen peroxide solution. The hydrogen peroxide solution may be an aqueous solution comprising hydrogen peroxide and water. As noted above, the hydrogen peroxide solution may have a weight ratio of hydrogen peroxide to water of about 10:1 to about 1:50, about 10:1 to about 1:10, or about 1:2 to about 2:1. The hydrogen peroxide solution may be added when the oral care composition or a base component thereof has a temperature of about 30° C. or less, about 28° C. or less, about 26° C. or less, or about 24° C. or less. In at least one embodiment, the hydrogen peroxide solution is added as the ultimate or penultimate component/ingredient into the oral care composition or a base component thereof.

According to another aspect, provided is a method for using the oral care compositions disclosed herein. In some embodiments, where the oral care composition of the disclosure is an oral care composition (e.g., a toothpaste), the oral care composition may be applied to a user's teeth according to typical means for brushing teeth. In some embodiments, the present invention provides a method of whitening a tooth surface; cleaning an oral cavity surface; or treating, preventing or ameliorating a disease, disorder or condition of the oral cavity, comprising: applying an effective amount of an oral care composition to an oral cavity surface of a subject in need thereof. The disease, disorder, or condition for which the oral care composition treats, prevents, or ameliorates is selected from: gingivitis; periodontitis; excessive plaque and/or tartar build-up; caries; tooth decay; stained teeth (e.g., intrinsic or extrinsic stains); halitosis; erosion; sensitivity; inflammation; and a combination of two or more thereof.

According to yet a further aspect of the invention, provided is a kit. The kit may include a container and an oral care composition disposed in the container. The oral care compositions can be packaged into containers or dispensers known in the art, via means conventional in the art. In some embodiments the compositions are packaged into tubes, metal, plastic or laminated, with either screw top or flip top caps. The container may be compatible with or contain a pump.

Embodiments of the present invention will now be further described by way of the following, non-limiting, examples.

### EXAMPLES

### Example 1

Four exemplary, non-limiting oral care compositions (Ex. A to Ex. D) are prepared in accordance with the formulations shown in Table 1, below. Ex. A to Ex. D are each in the form of a toothpaste composition:

TABLE 1

| | Ex. A | Ex. B | Ex. C | Ex. D | Ex. E |
|---|---|---|---|---|---|
| Ingredient | Wt. % | | | | |
| PROPYLENE GLYCOL | 40-70 | 40-60 | 40-60 | 40-60 | 40-60 |
| HYDROGEN PEROXIDE | 0.2 | 2 | 4 | 6 | 8 |
| Solution (50 wt. % Hydrogen | | | | | |
| peroxide and 50 wt. % water) | | | | | |
| POLYVINYL PYRROLIDONE | 1-15 | 1-15 | 1-15 | 1-15 | 1-15 |
| SODIUM LAURYL SULFATE | 1-5 | 1-5 | 1-5 | 1-5 | 1-5 |
| SILICON DIOXIDE | 0.1-10 | 0.1-10 | 0.1-10 | 0.1-10 | 0.1-10 |
| CALCIUM PYROPHOSPHATE | 10-30 | 10-30 | 10-30 | 10-30 | 10-30 |
| TETRASODIUM | 0.5-4 | 0.5-4 | 0.5-4 | 0.5-4 | 0.5-4 |
| PYROPHOSPHATE | | | | | |
| SODIUM ACID | 0.1-1 | 0.1-1 | 0.1-1 | 0.1-1 | 0.1-1 |
| PYROPHOSPHATE | | | | | |
| FLUORIDE ION SOURCE | 0.1-2 | 0.1-2 | 0.1-2 | 0.1-2 | 0.1-2 |
| PEG/PPG-116/66 Copolymer | 5-15 | 5-15 | 5-15 | 5-15 | 5-15 |
| Flavoring Agents | 0.1-4 | 0.1-4 | 0.1-4 | 0.1-4 | 0.1-4 |
| Preservative(s) | — | 0.03 | 0.03 | 0.03 | 0.03 |
| Total | 100 | 100 | 100 | 100 | 100 |

### Example 2

Example Compositions B and C (Ex. B and Ex. C) are studied under elevated temperatures to evaluate the stability of the hydrogen peroxide contained therein. Specifically, Ex. B and Ex. C are stored at a temperature of 40° C. for 4 weeks. The amount of hydrogen peroxide in Ex. B and Ex. C is evaluated after 1 week, after 2 weeks, after 3 weeks, and after 4 weeks of storage at a temperature of 40° C.

Ex. B and Ex. C are both determined to be stable. Additionally, the amount of active oxygen level, which is proportional to the amount of hydrogen peroxide, at the end of the study is determined, as shown in Table 2, below:

TABLE 2

| | Active Oxygen level after 40° C. |
|---|---|
| | aging |
| Example Composition B (1% HP) | 0.95% after 4 weeks |
| Example Composition C (2% HP) | 1.9% after 6 weeks |

### Example 3

Six exemplary, non-limiting oral care compositions (Ex. F to Ex. K) are prepared in accordance with the formulations shown in Table 3 (below). Certain compositions are evaluated to determine the amount of active oxygen level according to the procedures described in Example 2:

TABLE 3

| | Ex. F | Ex. G | Ex. H | Ex. I | Ex. J | Ex. K |
|---|---|---|---|---|---|---|
| Ingredient | Wt. % | | | | | |
| PROPYLENE GLYCOL | 40-65 | 40-60 | 40-60 | 40-65 | | 35-75 |
| POLYOXYPROPYLENE- | | | | | 40-70 | |
| POLYOXYETHYLENE BLOCK | | | | | | |
| COPOLYMER | | | | | | |
| HYDROGEN PEROXIDE Solution (50 | 0.2-10 | 0.2-10 | 0.2-10 | 0.2-10 | 0.2-10 | 0.2-10 |
| wt. % Hydrogen peroxide and 50 wt. % | | | | | | |
| water) | | | | | | |
| FUMED SILICA | — | 5-15 | — | — | — | — |
| CETEARYL ALCOHOL | — | — | — | 1-10 | — | — |
| POLYVINYL PYRROLIDONE | 10-20 | — | — | — | — | — |
| SODIUM STEARATE | — | — | 1-10 | — | 1-5 | 1-10 |
| POLYETHYLENE GLYCOL/ | 5-15 | 5-15 | 5-15 | 5-15 | 5-15 | — |
| POLYPROPYLENE GLYC-L1220 | | | | | | |
| FLUORIDE SOURCE | 0.1-1 | 0.1-1 | 0.1-1 | 0.1-1 | 0.1-1 | 0.1-1 |
| CALCIUM PYROPHOSPHATE | 5-20 | 5-20 | 5-20 | 5-20 | 5-20 | 5-20 |
| SODIUM LAURYL SULFATE | 0.1-5 | 0.1-5 | 0.1-5 | 0.1-5 | 0.1-5 | 0.1-5 |
| TETRASODIUM PYROPHOSPHATE | 0.1-4 | 0.1-4 | 0.1-4 | 0.1-4 | 0.1-4 | 0.1-4 |
| SODIUM ACID PYROPHOSPHATE | 0.1-2 | 0.1-2 | 0.1-2 | 0.1-2 | 0.1-2 | 0.1-2 |
| Flavoring Agents | 0.1-5 | 0.1-5 | 0.1-5 | 0.1-5 | 0.1-5 | 0.1-5 |
| Total | 100 | 100 | 100 | 100 | 100 | 100 |
| Active oxygen level after | 1.9 | 1.8 | 1.7 | NA | NA | NA |
| 60° C. for 7 days | | | | | | |

### Example 4

To test the effects of abrasive systems on stability, formulas containing 4% hydrogen peroxide (8% hydrogen peroxide solution) are examined with and without the addition of high cleaning silica. Formulas are aged at 60 degree C. for 1 week. The results of the remaining active oxygen levels are as follow:

TABLE 4

| | Ex. E | Ex. L | Ex. M |
|---|---|---|---|
| Ingredient | Wt. % | | |
| PROPYLENE GLYCOL | 40-60 | 40-60 | 40-60 |
| HYDROGEN PEROXIDE | 8 | 8 | 8 |
| Solution (50 wt. % Hydrogen | | | |
| peroxide and 50 wt. % water) | | | |
| POLYVINYL PYRROLIDONE | 1-15 | 1-15 | 1-15 |
| SODIUM LAURYL SULFATE | 1-5 | 1-5 | 1-5 |
| High Cleaning Silica | 0 | 5 | 10 |
| SILICON DIOXIDE | 0.1-10 | 0.1-10 | 0.1-10 |
| CALCIUM PYROPHOSPHATE | 10-30 | 10-30 | 10-30 |
| TETRASODIUM | 0.5-4 | 0.5-4 | 0.5-4 |
| PYROPHOSPHATE | | | |
| SODIUM ACID | 0.1-1 | 0.1-1 | 0.1-1 |
| PYROPHOSPHATE | | | |
| FLUORIDE ION SOURCE | 0.1-2 | 0.1-2 | 0.1-2 |
| PEG/PPG-116/66 Copolymer | 5-15 | 5-15 | 5-15 |
| Flavoring Agents | 0.1-4 | 0.1-4 | 0.1-4 |
| Preservative(s) | 0.03 | 0.03 | 0.03 |
| Total | 100 | 100 | 100 |

| | | Active Oxygen % after |
|---|---|---|
| Formula | | 60° C. after 1 week |
| E | 4% HP*, no high cleaning silica | 95.29% |
| L | 4% HP, 5% high cleaning silica | 14.46% |
| M | 4% HP, 10% high cleaning silica | 6.34% |

*The amount of hydrogen peroxide (HP) is listed as the active weight.

From the data in Table 4 a small amount of high cleaning silica may be associated with a loss of hydrogen peroxide in the formula. To achieve good peroxide stability, it is essential to have a formula backbone with high purity material that is compatible with hydrogen peroxide.

### Example 5

The stability, whitening efficacy, and flavor are evaluated for formulations containing a hydrogen peroxide solution as compared to market-based formulas with equivalent amounts of active hydrogen peroxide. Notably, the market-based formulas do not contain hydrogen peroxide solution. Rather, the market-based formulas are anhydrous and contain hydrogen peroxide-polyvinyl pyrrolidone polymer complexes.

Stability

TABLE 5

| | 40° C. | | 40° C. | | 40° C. | |
|---|---|---|---|---|---|---|
| | 1 month | | 2 months | | 3 months | |
| | Liquid | Market- | Liquid | Market- | Liquid | Market- |
| | HP | Based | HP | Based | HP | Based |
| 1% HP | 1.0% | 1.03% | 0.96% | 0.92% | 0.94% | 0.89% |
| | (Ex. B) | | (Ex. B) | | (Ex. B) | |
| 2% HP | 2.0% | 1.90% | 2.0% | 1.90% | 1.9% | 1.80% |
| | (Ex. C) | | (Ex. C) | | (Ex. C) | |
| 3% HP | 3.1% | 2.9% | 3.0% | 2.9% | 2.8% | 2.8% |
| | (Ex. D) | | (Ex. D) | | (Ex. D) | |
| 4% HP | 4.1% | 4.1% | 4.0% | 4.1% | 3.8% | 3.9% |
| | (Ex. E) | | (Ex. E) | | (Ex. E) | |

The formulas in Table 5 are subject to aging studies at 40° C. over the course of one, two, and three months. As seen in Table 5, despite the presence of water, the stability of the hydrogen peroxide, that derives hydrogen peroxide from the liquid solution, is comparable to anhydrous market-based formulas. This trend is maintained over the course of three months.

Whitening Efficacy and Flavor

In a separate assay, the whitening efficacy of formulas that incorporate hydrogen peroxide solution at: 1% hydrogen peroxide (Ex. B), 2% hydrogen peroxide (Ex. C), 3% hydrogen peroxide, and 4% hydrogen peroxide (Ex. E) perform at parity with anhydrous market-based formulas that incorporate hydrogen peroxide-polyvinyl pyrrolidone polymer complexes.

Toothpaste formulas that incorporate liquid hydrogen peroxide solution have equivalent whitening efficacy as market-based formulas that incorporate hydrogen peroxide via hydrogen peroxide-polyvinyl pyrrolidone polymer complexes.

Additionally, in a separate assay, the formulas that incorporate 4% and 3% hydrogen peroxide solution demonstrate improved flavor satisfaction relative to anhydrous market-based that incorporate hydrogen peroxide-polyvinyl pyrrolidone polymer complexes. Additionally, formulas that incorporate 4%, 3%, and 2% hydrogen peroxide solution demonstrate comparable whitening satisfaction relative to anhydrous market-based that incorporate hydrogen peroxide-polyvinyl pyrrolidone polymer complexes.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

1-42. (canceled)

43. An oral care composition comprising:
from about 35 wt. % to about 85 wt. % of an anhydrous solvent system;
from about 0.02 wt. % to about 30 wt. % of a liquid peroxide solution comprising:
(i) from about 0.01 wt. % to about 15 wt. % of hydrogen peroxide, and
(ii) from about 1 wt. % to about 15 wt. % of water,
wherein the hydrogen peroxide and water are present in a weight ratio of the hydrogen peroxide to the water of about 1:3 to about 2:1; and
from about 1 wt. % to about 20 wt. % of a thickening system, the thickening system consisting of a polyvinyl pyrrolidone polymer, a polyethylene glycol/polypropylene glycol copolymer, and optionally a silica thickener,
wherein the oral care composition has a total water content of less than about 30 wt. %, and all weight percentages are based on the total weight of the oral care composition.

44. The oral care composition according to claim 43, wherein the oral care composition comprises a about 1 wt. % or less of a fatty amphiphile.

45. The oral care composition according to claim 43, wherein the oral care composition is free of a fatty amphiphile.

46. The oral care composition according to claim 43, wherein the oral care composition has a weight ratio of the hydrogen peroxide to the polyvinyl pyrrolidone about 1:1 to about 1:8.

47. The oral care composition according to claim 43, wherein the weight ratio of total water content to polymer is from about 1:1 to about 1:10.

48. The oral care composition according to claim 43, wherein the anhydrous solvent system comprises a solvent selected from propylene glycol, butylene glycol, hexylene glycol, pentylene glycol, 1,3-propanediol, diethylene glycol, polyethylene glycol, dipropylene glycol, caprylyl glycol, glycerin, and a combination of two or more thereof.

49. The oral care composition according to claim 43 further comprising an abrasive selected from a silica, an alumina, an insoluble phosphate, calcium pyrophosphate, calcium carbonate, a resinous abrasive, and a combination of two or more thereof.

50. The oral care composition according to claim 49, wherein the abrasive comprises calcium pyrophosphate.

51. The oral care composition according to claim 43, wherein the oral care composition has a pH of from about 4.5 to about 9.

52. The oral care composition according to claim 43 further comprising sodium acid pyrophosphate in an amount from about 0.1 to about 1 wt. %.

53. An oral care composition comprising:
from about 35 wt. % to about 85 wt. % of an anhydrous solvent system;
from about 0.02 wt. % to about 30 wt. % of a liquid peroxide solution comprising:
(i) from about 0.01 wt. % to about 15 wt. % of hydrogen peroxide, and
(ii) from about 1 wt. % to about 15 wt. % of water,
wherein the hydrogen peroxide and water are present in a weight ratio of the hydrogen peroxide to the water of about 1:3 to about 2:1; and
from about 1 wt. % to about 20 wt. % of a thickening system,
wherein the oral care composition has a total water content of less than about 30 wt. %, and all weight percentages are based on the total weight of the oral care composition.

54. The oral care composition according to claim 53, wherein the oral care composition is free of peroxide sources other than the hydrogen peroxide solution.

55. The oral care composition according to claim 53, wherein the oral care composition is free of a fatty amphiphile.

56. The oral care composition according to claim 53, wherein the oral care composition has a weight ratio of the hydrogen peroxide to the polyvinyl pyrrolidone about 1:1 to about 1:8.

57. The oral care composition according to claim 53, wherein the weight ratio of total water content to polymer is from about 1:1 to about 1:10.

58. The oral care composition according to claim 53, wherein the thickening system comprises a thickening agent selected from a polymer, fumed silica, sodium stearate, stearic acid, and a combination of two or more thereof.

59. The oral care composition according to claim 53, wherein the thickening system consists of a polyvinyl pyrrolidone polymer, a polyethylene glycol/polypropylene glycol copolymer, and optionally a silica thickener.

60. The oral care composition according to claim 53 further comprising an abrasive selected from a silica, an alumina, an insoluble phosphate, calcium pyrophosphate, calcium carbonate, a resinous abrasive, and a combination of two or more thereof.

61. The oral care composition according to claim 59, wherein the abrasive comprises calcium pyrophosphate.

62. The oral care composition according to claim 59, wherein the abrasive consists of calcium pyrophosphate, fumed silica, or a combination of two or more thereof.
